(19) United States
(12) Patent Application Publication
(10) Pub. No.: US 2023/0174497 A1
(43) Pub. Date: Jun. 8, 2023

(54) RHEIN AMIDE DERIVATIVE AND PREPARATION METHOD AND USE THEREOF, AND INHIBITOR OF HEPATOCELLULAR CARCINOMA (HCC) WITH SPECIFIC RECQL4 EXPRESSION

(71) Applicant: Shandong First Medical University & Shandong Academy of Medical Sciences, Jinan City (CN)

(72) Inventors: Xuemei ZHAO, Jinan City (CN); Zhuo WANG, Jinan City (CN); Guiling WANG, Jinan City (CN); Xuelian ZHANG, Jinan City (CN); Jialing WANG, Jinan City (CN); Linlin FU, Jinan City (CN); Weihua DI, Jinan City (CN)

(21) Appl. No.: 17/811,201

(22) Filed: Jul. 7, 2022

(30) Foreign Application Priority Data
Dec. 7, 2021 (CN) 202111485915.2

(51) Int. Cl. C07D 295/215; C07C 233/87; C07C 233/65

(52) U.S. Cl. CPC C07D 295/215; C07C 233/65; C07C 233/87

(57) ABSTRACT

A rhein amide derivative and a preparation method and use thereof, and a drug for treating hepatocellular carcinoma (HCC) caused by a specific expression of RECQL4. The rhein amide derivative has a structure shown in any one of formulas I to IV. The rhein amide derivative has a relatively strong inhibitory effect on proliferation of HCC cells SNU-398 with RECQL4 high expression. Development of a novel anti-cancer drug for RECQL4 high expression-caused HCC and clinical personalized treatment of the RECQL4 high expression-caused HCC.

### CROSS REFERENCE TO RELATED APPLICATION

This application claims priority to Chinese Patent Application No. 202111485915.2, filed on Dec. 7, 2021, the disclosure of which is incorporated by reference herein in its entirety..

### TECHNICAL FIELD

The present disclosure belongs to the technical field of organic synthetic drugs, in particular to a rhein amide derivative and a preparation method and use thereof, and an inhibitor of hepatocellular carcinoma (HCC) with specific RECQL4 expression.

### BACKGROUND ART

Hepatocellular carcinoma (HCC) is a primary liver malignancy. Studies have shown that the causes of HCC are complex, and the number of patients and deaths is increasing annually. In addition to ethnic and genetic background, the main causes of HCC development vary worldwide. Hepatitis virus, fatty liver, autoimmune liver diseases, intake of a fungal metabolite aflatoxin B1, DNA damage may lead to the occurrence of HCC. However, genomic instability is a major feature of cancer development. RECQ helicase, as a member of the DNA helicase family, plays an important role in maintaining gene stability. The RECQ helicase family includes five proteins, RECQ1, WRN, BLM, RECQL4, and RECQ. Abnormally low expression of the RECQL4 is associated with cancer susceptibility and premature aging, and upregulation of the RECQL4 is associated with cancer initiation and metastasis. In vitro experiments show that the RECQL4 can act synergistically with the BLM to increase helicase activity in an S phase of the cell cycle and increase nuclear genome stability. RECQL4 can regulate the normal function of chromosomes, improve the stability of mitochondria, and maintain the normal state of human genes. Abnormal expression or deletion of the RECQL4 may significantly increase a level of reactive oxygen species in cells, and greatly reduce a repair capacity of oxidative damages in mitochondrial DNA. This suggests that human RECQL4 plays an important role in the initiation of DNA replication.

Therefore, in HCC patients with up-regulated RECQL4 expression, silencing or inhibiting the function of RECQL4, inhibiting the proliferation of tumor cells, and promoting apoptosis of tumor cells may also be a new anticancer therapy. Targeted control of RECQL4 protein expression can play a role in the treatment of HCC.

At present, there is no report on screening inhibitors that inhibit HCC cell proliferation using the RECQL4 as a target.

### SUMMARY

In view of this, the present disclosure provides a rhein amide derivative and a preparation method and use thereof, and an inhibitor of HCC with specific RECQL4 expression. The rhein amide derivative has a relatively strong inhibitory effect on proliferation of HCC cells with high RECQL4 expression.

To solve the above technical problems, the present disclosure provides a rhein amide derivative, having a structure shown in any one of formulas I to IV:

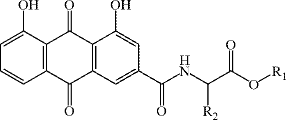

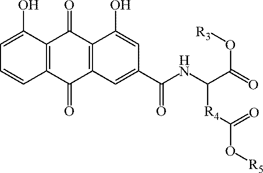

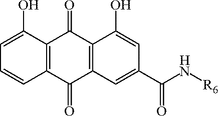

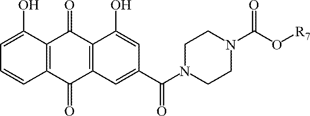

R₁, R₂, R₃, R₄, R₅, and R₇ are independently selected from the group consisting of methyl and ethyl; and R₆ is linear C₁₋₄ alkyl.

Preferably, the rhein amide derivative may have a structure shown in formula 1-1, formula 1-2, formula II-1, formula II-2, formula III-1, formula III-2, or formula IV-1:

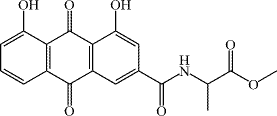 [SMILES: COC(=O)C(C)NC(=O)c1cc(O)c2c(c1)C(=O)c1cccc(O)c1C2=O]

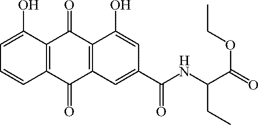 [SMILES: CCOC(=O)C(CC)NC(=O)c1cc(O)c2c(c1)C(=O)c1cccc(O)c1C2=O]

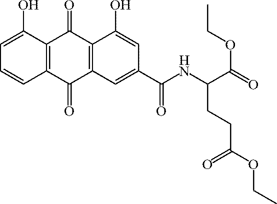 [SMILES: CCOC(=O)CCC(NC(=O)c1cc(O)c2c(c1)C(=O)c1cccc(O)c1C2=O)C(=O)OCC]

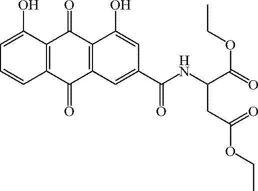 [SMILES: CCOC(=O)CC(NC(=O)c1cc(O)c2c(c1)C(=O)c1cccc(O)c1C2=O)C(=O)OCC]

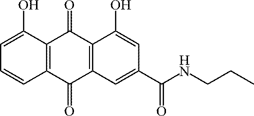 [SMILES: CCCNC(=O)c1cc(O)c2c(c1)C(=O)c1cccc(O)c1C2=O]

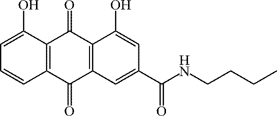 [SMILES: CCCCNC(=O)c1cc(O)c2c(c1)C(=O)c1cccc(O)c1C2=O]

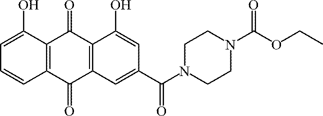 [SMILES: CCOC(=O)N1CCN(C(=O)c2cc(O)c3c(c2)C(=O)c2cccc(O)c2C3=O)CC1]

The present disclosure further provides a preparation method of the rhein amide derivative, including the following steps:
mixing rhein, any one of a reactant 1 to a reactant 3, a coupling agent, an acylation catalyst, an organic base, and an organic solvent to conduct an amidation reaction, to obtain the rhein amide derivative having a structure shown in any one of formulas I to III; and
mixing the rhein, a reactant 4, the coupling agent, the acylation catalyst, and the organic solvent to conduct the amidation reaction, to obtain a rhein amide derivative having a structure shown in formula IV; where
the reactant 1 is a hydrochloride of

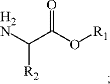

the reactant 2 is a hydrochloride of

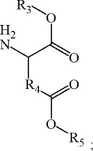

the reactant 3 is

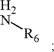

and the reactant 4 is

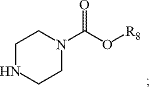

and
R₁, R₂, R₃, R₄, R₅, and R₇ are independently selected from the group consisting of methyl and ethyl; and R₆ is linear C₁₋₄ alkyl.

Preferably, the rhein and any one of the reactant 1 to the reactant 4 may have a molar ratio of 1:(1.0-1.5).

Preferably, the rhein and the coupling agent may have a molar ratio of 1:(1.8-2.5).

Preferably, the rhein and the acylation catalyst may have a molar ratio of 1:(1.8-2.3).

Preferably, the rhein and the organic base may have a molar ratio of 1:(2.5-4.5).

Preferably, the amidation reaction may be conducted at room temperature for 1.8 h to 7 h.

The present disclosure further provides use of the rhein amide derivative or a rhein amide derivative prepared by the preparation method in preparation of a drug for treating HCC caused by overexpression of RECQL4.

The present disclosure further provides an inhibitor for inhibiting overexpression of RECQL4, including a rhein amide derivative, where the rhein amide derivative includes the rhein amide derivative described in the above technical scheme or a rhein amide derivative prepared by the preparation method described in the above technical scheme.

The present disclosure provides a rhein amide derivative, having a structure shown in any one of formulas I to IV:

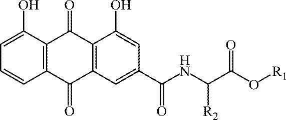

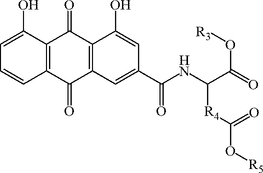

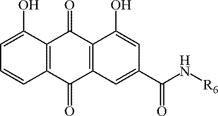

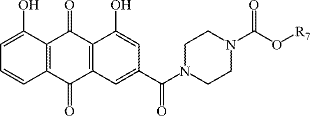

R₁, R₂, R₃, R₄, R₅, and R₇ are independently selected from the group consisting of methyl and ethyl; and R₆ is linear C₁₋₄ alkyl.

The rhein amide derivative has a relatively strong inhibitory effect on proliferation of HCC cells SNU-398 with RECQL4 high expression. Test results show that the rhein amide derivative provided in an example has a half maximal inhibitory concentration (IC₅₀) on HCC cells SNU398 smaller than that on hepatocytes LO2. This indicates that the rhein amide derivative has a relatively strong inhibitory effect on proliferation of the HCC cells SNU398 with high RECQL4 expression.

In the present disclosure, scientific experimental data is provided for development of a novel anti-cancer drug for RECQL4 high expression-caused HCC and clinical personalized treatment of the RECQL4 high expression-caused HCC.

### DETAILED DESCRIPTION OF THE EMBODIMENTS

The present disclosure provides a rhein amide derivative, having a structure shown in any one of formulas I to IV:

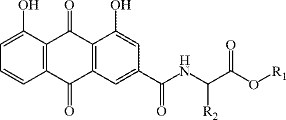

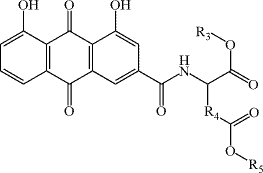

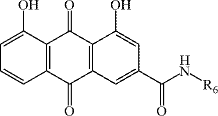

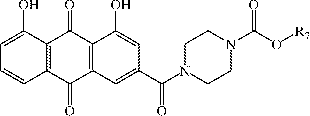

R₁, R₂, R₃, R₄, R₅, and R₇ are independently selected from the group consisting of methyl and ethyl; and R₆ is linear C₁₋₄ alkyl.

In the present disclosure, the rhein amide derivative has a structure preferably shown in formula 1-1, formula 1-2, formula II-1, formula II-2, formula III-1, formula III-2, or formula IV-1:

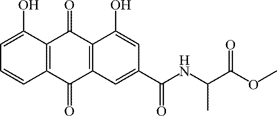 [SMILES: COC(=O)C(C)NC(=O)c1cc(O)c2c(c1)C(=O)c1cccc(O)c1C2=O]

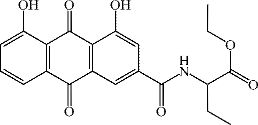 [SMILES: CCOC(=O)C(CC)NC(=O)c1cc(O)c2c(c1)C(=O)c1cccc(O)c1C2=O]

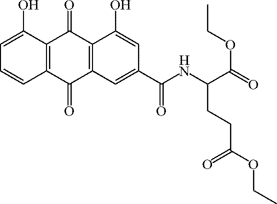 [SMILES: CCOC(=O)CCC(NC(=O)c1cc(O)c2c(c1)C(=O)c1cccc(O)c1C2=O)C(=O)OCC]

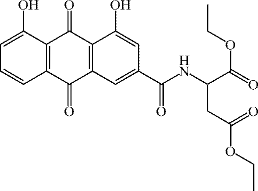 [SMILES: CCOC(=O)CC(NC(=O)c1cc(O)c2c(c1)C(=O)c1cccc(O)c1C2=O)C(=O)OCC]

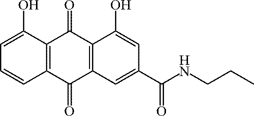 [SMILES: CCCNC(=O)c1cc(O)c2c(c1)C(=O)c1cccc(O)c1C2=O]

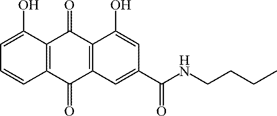 [SMILES: CCCCNC(=O)c1cc(O)c2c(c1)C(=O)c1cccc(O)c1C2=O]

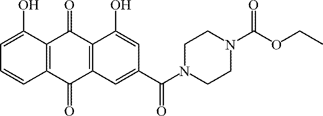 [SMILES: CCOC(=O)N1CCN(C(=O)c2cc(O)c3c(c2)C(=O)c2cccc(O)c2C3=O)CC1]

The present disclosure further provides a preparation method of the rhein amide derivative, including the following steps:
mixing rhein, any one of a reactant 1 to a reactant 3, a coupling agent, an acylation catalyst, an organic base, and an organic solvent (hereinafter referred to as a first mixing) to conduct an amidation reaction, to obtain the rhein amide derivative having a structure shown in any one of formulas I to V; and
mixing the rhein, a reactant 4, the coupling agent, the acylation catalyst, and the organic solvent (hereinafter referred to as a second mixing) to conduct the amidation reaction, to obtain a rhein amide derivative having a structure shown in formula III; where
the reactant 1 is a hydrochloride of

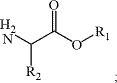

the reactant 2 is a hydrochloride of

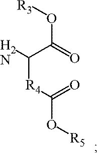

the reactant 3 is

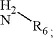

and the reactant 4 is

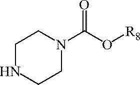

and
R₁, R₂, R₃, R₄, R₅, and R₇ are independently selected from the group consisting of methyl and ethyl; and R₆ is linear C₁₋₄ alkyl.

In the present disclosure, unless otherwise specified, the raw materials used are all commercially-available products well known to those skilled in the art.

In a specific example of the present disclosure, the reactant 1 is specifically preferably selected from the group consisting of alanine methyl ester hydrochloride and ethyl 2-aminobutyrate hydrochloride.

In a specific example of the present disclosure, the reactant 2 is specifically preferably selected from the group consisting of diethyl glutamate hydrochloride and diethyl aspartate hydrochloride.

In a specific example of the present disclosure, the reactant 3 is specifically preferably selected from the group consisting of n-propylamine and n-butylamine.

In a specific example of the present disclosure, the reactant 4 is specifically preferably 4-(1-piperazinyl) ethyl formate.

In a specific example of the present disclosure, the coupling agent is specifically preferably 1-ethyl-(3-dimethylaminopropyl) carbodiimide hydrochloride (EDC·HCl). The EDC· HCl can combine with -COOH in a rhein structure to form an unstable intermediate.

In a specific example of the present disclosure, the acylation catalyst is specifically preferably 1-hydroxybenzotriazole (HOBt). The HOBt reacts with the unstable intermediate to form an active ester.

In a specific example of the present disclosure, the organic base is specifically preferably triethylamine. The organic base provides an alkaline reaction environment for the amidation reaction.

In a specific example of the present disclosure, the organic solvent is specifically preferably dichloromethane.

In the present disclosure, the rhein and any one of the reactant 1 to the reactant 4 have a molar ratio of 1:(1.0-1.5), more preferably 1:1.2.

In the present disclosure, the rhein and the coupling agent have a molar ratio of preferably 1:(1.8-2.5), more preferably 1:2.

In the present disclosure, the rhein and the acylation catalyst have a molar ratio of preferably 1:(1.8-2.3), more preferably 1:2.

In the present disclosure, the rhein and the organic base have a molar ratio of preferably 1:(2.5-4.5), more preferably 1:4.

In the present disclosure, a mass of the rhein and a volume of the organic solvent have a ratio of preferably (1.6-5) mg: 1 mL.

In the present disclosure, the first mixing is conducted preferably by: mixing the rhein, the coupling agent, the acylation catalyst, any one of the reactants 1 to 3, the organic base, and the organic solvent in sequence.

In the present disclosure, the second mixing is conducted preferably by: mixing the rhein, the coupling agent, the acylation catalyst, the reactant 4, and the organic solvent in sequence.

In the present disclosure, the first mixing and the second mixing are independently conducted under stirring; there is no special requirement for a specific implementation process of the stirring.

In the present disclosure, an amidation reaction solution is obtained by the amidation reaction; preferably, the amidation reaction solution is subjected to post-treatment, to obtain the rhein amide derivative.

In a specific example of the present disclosure, when the reaction raw material is preferably the alanine methyl ester hydrochloride, the post-treatment includes preferably: conducting solid-liquid separation, washing a solid product, and drying sequentially. The solid-liquid separation is preferably filtration; preferably, the solid product obtained by the solid-liquid separation is washed; the washing preferably includes: washing with water, ethanol and then dichloromethane in sequence. The washing is preferably rinsing; a volume of water and a mass of the solid product during the washing have a ratio of preferably 250 mL: 1 g. A volume of the ethanol and a mass of the solid product have a ratio of preferably 50 mL: 1 g during the ethanol washing. A volume of the dichloromethane and a mass of the solid product have a ratio of preferably 10 mL: 1 g during the dichloromethane washing. Preferably, a washed solid product is dried; the drying is conducted at preferably 65° C. The drying is conducted for preferably 8 h.

In a specific example of the present disclosure, when the reaction raw material is specifically preferably the ethyl 2-aminobutyrate hydrochloride, the diethyl glutamate hydrochloride, the diethyl aspartate hydrochloride, the n-propylamine, the n-butylamine, N-methylpiperazine, or the 4-(1-piperazinyl) ethyl formate, the post-treatment includes preferably: mixing the amidation reaction solution with a silica gel powder; desolventizing a mixture to obtain a solid powder, conducting dry sample loading on the solid powder to conduct purification by silica gel column chromatography, collecting an eluent of a second color band, and drying the eluent to obtain the rhein amide derivative. A volume of the amidation reaction solution and a mass of the silica gel powder have a ratio of preferably 30 mL: 200 mg. The desolventizing is preferably conducted by rotary evaporation; there is no special requirement for a specific implementation process of the desolventizing by rotary evaporation. The purification by silica gel column chromatography is conducted by preferably dry sample loading; the eluent is preferably petroleum ether-ethyl acetate; an elution procedure is preferably petroleum ether: ethyl acetate of 12:1 to 4:1. An eluate is dried at preferably 65° C. for preferably 8 h to obtain the rhein amide derivative.

In the present disclosure, a structure of the rhein amide derivative is identified preferably by hydrogen nuclear magnetic resonance (¹H NMR), carbon nuclear magnetic resonance (¹³C NMR), and high-resolution mass spectrometry (HRMS).

The present disclosure further provides use of the rhein amide derivative or a rhein amide derivative prepared by the preparation method in preparation of a drug for treating HCC caused by overexpression of RECQL4.

The present disclosure further provides an inhibitor for inhibiting overexpression of RECQL4, including a rhein amide derivative, where the rhein amide derivative includes the rhein amide derivative described in the above technical scheme or a rhein amide derivative prepared by the preparation method described in the above technical scheme.

In order to further illustrate the present disclosure, the technical solutions provided by the present disclosure are described in detail below in connection with examples, but these examples should not be understood as limiting the claimed scope of the present disclosure.

### Example 1

90 mg of rhein was added to a 50 ml round bottom flask; EDC · HC1 (a molar ratio of the EDC · HC1 to the rhein was 2), HOBt (a molar ratio of the HOBt to the rhein was 2), alanine methyl ester hydrochloride (a molar ratio of the alanine methyl ester hydrochloride to the rhein was 1.2), triethylamine (a molar ratio of the triethylamine and the rhein was 4), and 30 mL of dichloromethane as a reaction solvent were added, and stirred to dissolve; an amidation reaction was conducted for 4 h at room temperature, and a yellow precipitate was formed; an amidation reaction solution was filtered with filter paper, a solid product was washed with water, absolute ethanol and then dichloromethane in sequence, and dried; a structure was identified by ¹H NMR, ¹³C NMR and HRMS, and a rhein amide derivative shown in formula I-1 was obtained as: methyl(4,5-dihydroxy-9,10-dioxy-9,10-dihydroanthracene-2-carbonyl) alaninate, which was a yellow solid, with a yield (70 mg, 60%) and a melting point of 236.3° C. to 237.9° C.

¹H NMR (400 MHz, DMSO-d₆) δ 11.96 (s, 2H), 9.49 (t, J = 5.8 Hz, 1H), 8.20 (d, J = 1.7 Hz, 1H), 7.93 - 7.85 (m, 1H), 7.85 - 7.78 (m, 2H), 7.46 (dt, J = 8.5, 1.7 Hz, 1H), 4.13 (d, J = 5.8 Hz, 1H), 3.74 (s, 3H), 1.27 - 1.16 (m, 3H).

¹³C NMR (101 MHz, DMSO-d₆) δ 191.92, 181.57, 170.49, 164.98, 161.86, 161.61, 141.11, 138.07, 134.24, 133.76, 125.05, 122.96, 119.94, 118.39, 117.92, 116.59, 52.37, 41.83, 39.60, 39.37.

HRMS (ESI+): molecular formula C₁₉H₁₅NO₇, [M+H]⁺370.0927, measured value: 370.0936.

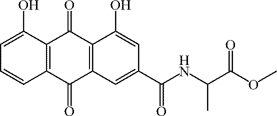 [SMILES: COC(=O)C(C)NC(=O)c1cc(O)c2c(c1)C(=O)c1cccc(O)c1C2=O]

### Example 2

90 mg of rhein was added to a 50 ml round bottom flask; EDC · HC1 (a molar ratio of the EDC · HC1 to the rhein was 2), HOBt (a molar ratio of the HOBt to the rhein was 2), ethyl 2-aminobutyrate hydrochloride (a molar ratio of the ethyl 2-aminobutyrate hydrochloride to the rhein was 1.2), triethylamine (a molar ratio of the triethylamine and the rhein was 4), and 30 mL of dichloromethane as a reaction solvent were added, and stirred to dissolve; an amidation reaction was conducted for 4 h at room temperature, 200 mg of a silica gel powder was added, and the solvent was evaporated to dryness on a rotary evaporator to obtain a yellow-red powder. Elution was conducted by silica gel column chromatography, an eluent of a second color band was collected, the eluent was concentrated and dried to obtain a yellow powdery solid, and a structure was identified by ¹H NMR, ¹³C NMR and HRMS to obtain a rhein amide derivative shown in formula I-2 as: ethyl 2-(4,5-dihydroxy-9,10-dioxy-9,10-dihydroanthracene-2-carboxamide)butanoate, which was a yellow solid, with a yield (105 mg, 83.7%) and a melting point of 196.7° C. to 197.2° C.

¹H NMR (400 MHz, DMSO-d₆) δ 11.89 (d, J = 9.5 Hz, 2H), 9.17 (d, J = 7.2 Hz, 1H), 8.16 (d, J = 1.7 Hz, 1H), 7.86 - 7.79 (m, 2H), 7.74 (dd, J = 7.5, 1.2 Hz, 1H), 7.40 (dd, J = 8.4, 1.2 Hz, 1H), 4.36 (ddd, J = 9.0, 7.2, 5.5 Hz, 1H), 4.20 - 4.11 (m, 2H), 1.92 - 1.79 (m, 2H), 1.22 (t, J = 7.1 Hz, 3H), 0.99 (t, J = 7.4 Hz, 3H).

¹³C NMR (101 MHz, DMSO-d₆) δ 191.97, 172.24, 165.10, 161.87, 161.56, 141.42, 138.06, 133.77, 125.01, 123.19, 119.92, 118.27, 116.57, 61.02, 55.09, 24.27, 14.59, 11.25.

HRMS (ESI+): molecular formula C₂₁H₁₉NO₇, [M+H]⁺398.1240, measured value: 398.1255.

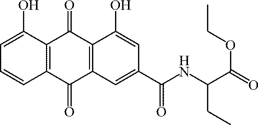 [SMILES: CCOC(=O)C(CC)NC(=O)c1cc(O)c2c(c1)C(=O)c1cccc(O)c1C2=O]

### Example 3

Basically the same as a preparation method of Example 2, the difference was: a raw material was diethyl glutamate hydrochloride. A rhein amide derivative shown in formula II-1 was obtained as: diethyl(4,5-dihydroxy-9,10-dioxo-9,10-dihydroanthracene-2-carbonyl) glutamate, which was a yellow solid, with a yield (85 mg, 75.3%) and a melting point of 202.0° C. to 202.3° C.

¹H NMR (400 MHz, DMSO-d₆) δ 11.83 (s, 2H), 9.19 (d, J = 7.3 Hz, 1H), 8.11 (d, J = 1.7 Hz, 1H), 7.83 - 7.75 (m, 2H), 7.69 (dd, J = 7.6, 1.2 Hz, 1H), 7.36 (dd, J = 8.4, 1.2 Hz, 1H), 4.70 - 4.37 (m, 1H), 4.20 - 4.02 (m, 4H), 2.48 (d , J = 7.5 Hz, 2H), 2.21 - 1.99 (m, 2H), 1.23 - 1.15 (m, 6H).

¹³C NMR (101 MHz, DMSO-d₆) δ 191.86, 181.42, 172.67, 171.85, 164.97, 161.85, 161.56, 141.20, 138.04, 134.00, 133.64, 125.00, 123.17, 119.91, 118.19, 118.16, 116.41, 61.25, 60.47, 52.76, 30.62, 26.04, 14.53 (d).

HRMS (ESI+): molecular formula C₂₄H₂₃NO₉, [M+H]⁺470.1451, measured value: 470.1468.

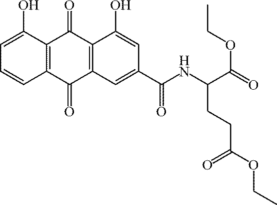 [SMILES: CCOC(=O)CCC(NC(=O)c1cc(O)c2c(c1)C(=O)c1cccc(O)c1C2=O)C(=O)OCC]

### Example 4

Basically the same as a preparation method of Example 2, the difference was: a raw material was diethyl aspartate hydrochloride. A rhein amide derivative shown in formula II-2 was obtained as: diethyl(4,5-dihydroxy-9,10-dioxo-9,10-dihydroanthracene-2-carbonyl)aspartate, which was a yellow solid, with a yield (58 mg, 40.3%) and a melting point of 174.7° C. to 175.2° C.

¹H NMR (400 MHz, DMSO-d₆) δ 11.84 (s, 2H), 9.38 (d, J= 7.6 Hz, 1H), 8.09 (d, J= 1.5 Hz, 1H), 7.79 (t, J = 7.9 Hz, 1H), 7.76 - 7.66 (m, 2H), 7.36 (d, J = 8.3 Hz, 1H), 4.89 (q, J = 7.3 Hz, 1H), 4.34 - 3.97 (m, 4H), 3.08 - 2.84 (m, 2H), 1.23 (t, J = 7.1 Hz, 6H).

¹³C NMR (101 MHz, DMSO-d₆) δ 191.78, 181.34, 170.81, 170.42, 164.50, 161.84, 161.54, 141.07, 138.03, 134.02, 133.55, 125.00, 123.06, 119.90, 117.97, 116.34, 61.57, 60.88, 50.03, 35.88, 14.49, 14.45.

HRMS (ESI+): molecular formula C₂₃H₂₁NO₉, [M+H]⁺456.1295, measured value: 456.1298.

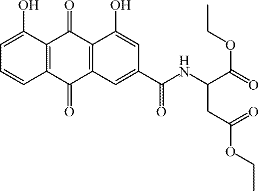 [SMILES: CCOC(=O)CC(NC(=O)c1cc(O)c2c(c1)C(=O)c1cccc(O)c1C2=O)C(=O)OCC]

### Example 5

Basically the same as a preparation method of Example 2, the difference was: a raw material was n-propylamine. A rhein amide derivative shown in formula III-1 was obtained as: 4,5-dihydroxy-9,10-dioxo-n-propyl-9,10-dihydroanthracene-2-carboxamide, which was a yellow solid, with a yield (28 mg, 27.3%) and a melting point of 264.1° C. to 265.0° C.

¹H NMR (400 MHz, DMSO-d₆) δ 11.91 (s, 2H), 8.91 (t, J = 5.6 Hz, 1H), 8.14 (d, J = 1.6 Hz, 1H), 7.84 (dd, J = 8.4, 7.5 Hz, 1H), 7.79 - 7.74 (m, 2H), 7.42 (dd, J = 8.3, 1.2 Hz, 1H), 3.29 - 3.24 (m, 2H), 1.67 - 1.50 (m, 2H), 0.91 (t, J = 7.4 Hz, 3H).

¹³C NMR (101 MHz, DMSO-d₆) δ 164.38, 161.87, 161.65, 134.09, 133.85, 125.02, 122.86, 119.93, 118.07, 116.62, 41.74, 22.64, 11.94.

HRMS (ESI+): molecular formula C₁₈H₁₅NO₅, [M+H]⁺326.1028, measured value: 326.1034.

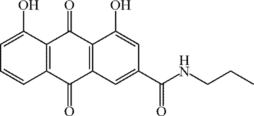 [SMILES: CCCNC(=O)c1cc(O)c2c(c1)C(=O)c1cccc(O)c1C2=O]

### Example 6

Basically the same as a preparation method of Example 2, the difference was: a raw material was n-butylamine. A rhein amide derivative shown in formula III-2 was obtained as: N-butyl-4,5-dihydroxy-9,10-dioxy-9,10-dihydroanthracene-2-carboxamide, which was a yellow solid, with a yield (35 mg, 33.3%) and a melting point of 215.2° C. to 215.9° C.

¹H NMR (400 MHz, DMSO-d₆) δ 11.90 (s, 2H), 8.88 (t, J = 5.5 Hz, 1H), 8.12 (d, J = 1.7 Hz, 1H), 7.83 (dd, J = 8.3, 7.5 Hz, 1H), 7.79 - 7.71 (m, 2H), 7.41 (dd, J = 8.3, 1.2 Hz, 1H), 3.32 - 3.26 (m, 2H), 1.62 - 1.49 (m, 2H), 1.39 - 1.28 (m, 2H), 0.92 (t, J = 7.3 Hz, 3H).

¹³C NMR (101 MHz, DMSO-d₆) δ 192.01, 181.63, 164.30, 161.86, 161.64, 142.38, 138.04, 134.05, 125.00, 122.85, 119.93, 118.07, 117.97, 116.58, 39.64, 31.44, 20.12, 14.18.

HRMS (ESI+): molecular formula C₁₉H₁₇NO₅, [M+H]⁺340.1185, measured value: 340.1193.

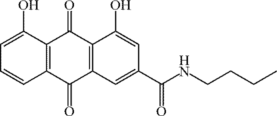 [SMILES: CCCCNC(=O)c1cc(O)c2c(c1)C(=O)c1cccc(O)c1C2=O]

### Example 7

Basically the same as a preparation method of Example 2, the difference was: a raw material was N-methylpiperazine without triethylamine. A rhein amide derivative shown in formula IV-1 was obtained as: ethyl (4-(4,5-dihydroxy-9,10-dioxy-9,10-dihydroanthracene-2-carbonyl)piperazin-1-yl)benzoate, which was a yellow solid, with a yield (28 mg, 37.5%) and a melting point of 222.2° C. to 222.8° C.

¹H NMR (400 MHz, Chloroform-d) δ 12.15 (s, 1H), 11.97 (s, 1H), 7.88 - 7.79 (m, 2H), 7.78 - 7.69 (m, 1H), 7.38 - 7.30 (m, 2H), 4.18 (qd, J = 7.2, 2.1 Hz, 2H), 3.79 (s, 2H), 3.62 (s, 2H), 3.45 (s, 4H), 1.26 (t, J = 2.5 Hz, 3H).

¹³C NMR (101 MHz, Chloroform-d) δ 192.63, 181.11, 167.30, 162.75, 162.68, 144.59, 137.61, 134.08, 133.43, 125.00, 122.50, 120.36, 118.00, 116.27, 115.76, 77.23, 55.19, 54.57, 47.50, 46.02, 42.13.

HRMS (ESI+): molecular formula C₂₂H₂₀N₂O₇, [M+H]⁺425.1349, measured value: 425.1358.

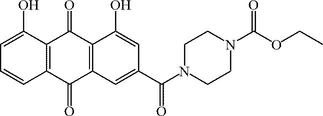 [SMILES: CCOC(=O)N1CCN(C(=O)c2cc(O)c3c(c2)C(=O)c2cccc(O)c2C3=O)CC1]

### Comparative Example 1

Basically the same as a preparation method of Example 1, the difference was: a raw material was methyl leucinate hydrochloride. A rhein amide derivative shown in formula V-1 was obtained as: methyl (4,5-dihydroxy-9,10-dioxo-9,10-dihydroanthracene-2-carbonyl)leucine, which was a yellow solid, with a yield (60 mg, 46%) and a melting point of 175.1° C. to 176.3° C.

¹H NMR (400 MHz, DMSO-d₆) δ 11.88 (s, 2H), 9.20 (d, J = 7.6 Hz, 1H), 8.15 (d, J = 1.7 Hz, 1H), 7.86 - 7.77 (m, 2H), 7.73 (dd, J = 7.5, 1.2 Hz, 1H), 7.39 (dd, J = 8.3, 1.2 Hz, 1H), 4.58 - 4.50 (m, 1H), 3.68 (s, 3H), 1.88 - 1.79 (m, 1H), 1.75 - 1.58 (m, 2H), 0.93 (dd, J = 17.0, 6.5 Hz, 6H).

¹³C NMR (101 MHz, DMSO-d₆) δ 191.90, 181.40, 172.36, 165.23, 161.86, 161.51, 141.42, 138.04, 133.88, 124.97, 123.36, 119.89, 118.49, 118.06, 58.09, 52.21, 36.07, 25.73, 15.95, 11.38.

HRMS (ESI+): molecular formula C₂₂H₂₁NO₇, [M+H]⁺412.1396, measured value: 412.1399.

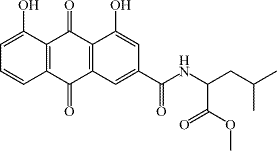 [SMILES: COC(=O)C(CC(C)C)NC(=O)c1cc(O)c2c(c1)C(=O)c1cccc(O)c1C2=O]

### Comparative Example 2

Basically the same as a preparation method of Example 1, the difference was: a raw material was methyl isoleucinate hydrochloride. A rhein amide derivative shown in formula V-2 was obtained as: methyl 2-(4,5-dihydroxy-9,10-dioxy-9,10-dihydroanthracene-2-carboxamide)-3-methylvalerate, which was a yellow solid, with a yield (35.1 mg, 37%) and a melting point of 179.7° C. to 182.3° C.

¹H NMR (400 MHz, DMSO-d₆) δ 11.85 (s, 2H), 9.11 (d, J = 7.5 Hz, 1H), 8.10 (d, J = , 1H), 7.84 - 7.76 (m, 2H), 7.69 (dd, J = 7.5, 1.2 Hz, 1H), 7.36 (dd, J = 8.4, 1.2 Hz, 1H), 4.40 (t, J = 7.5 Hz, 1H), 3.70 (s, 3H), 2.07 - 1.93 (m, 1H), 1.54 (dqd, J = 14.8, 7.4, 4.1 Hz, 1H), 1.34 -1.28 (m, 1H), 0.97 - 0.85 (m, 6H).

¹³C NMR (101 MHz, DMSO-d₆) δ 191.90, 181.40, 172.36, 165.23, 161.86, 161.51, 141.42, 138.04, 133.88, 133.66, 124.97, 123.36, 119.89, 118.49, 118.06, 58.09, 52.21, 36.07, 25.73, 15.95, 11.38.

HRMS (ESI+): molecular formula C₂₂H₂₁NO₇, [M+H]⁺412.1396, measured value: 412.1397.

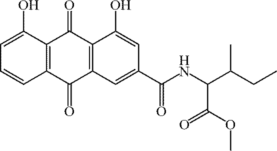 [SMILES: CCC(C)C(NC(=O)c1cc(O)c2c(c1)C(=O)c1cccc(O)c1C2=O)C(=O)OC]

### Comparative Example 3

Basically the same as a preparation method of Example 1, the difference was: a raw material was ethyl leucinate hydrochloride. A rhein amide derivative shown in formula V-3 was obtained as: ethyl(4,5-dihydroxy-9,10-dioxo-9,10-dihydroanthracene-2-carbonyl)leucinate, which was a yellow solid, with a yield (74 mg, 55%) and a melting point of 167.9° C. to 170.0° C.

¹H NMR (400 MHz, DMSO-d₆) δ 11.77 (d, J = 7.3 Hz, 2H), 9.10 (d, J = , 1H), 8.04 (d, J = 1.6 Hz, 1H), 7.75 - 7.68 (m, 2H), 7.61 (dd, J = 7.5, 1.2 Hz, 1H), 7.28 (dd, J = 8.4, 1.1 Hz, 1H), 4.63 - 4.32 (m, 1H), 4.27 - 3.97 (m, 2H), 1.81 - 1.62 (m, 2H), 1.60 - 1.52 (m, 1H), 1.16 (t, J = 7.1 Hz, 3H), 0.88 (dd, J = 17.4, 6.5 Hz, 6H).

¹³C NMR (101 MHz, DMSO-d₆) δ 191.85, 181.36, 172.70, 164.89, 161.85, 161.57, 141.34, 138.03, 133.96, 133.61, 124.97, 123.15, 119.89, 118.24, 118.07, 116.36, 61.12, 51.80, 39.58, 24.94, 23.29, 21.65, 14.56.

HRMS (ESI+): molecular formula C₂₃H₂₃NO₇, [M+H]⁺426.1553, measured value: 426.1580.

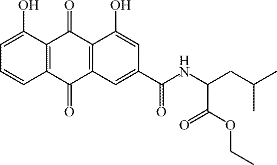 [SMILES: CCOC(=O)C(CC(C)C)NC(=O)c1cc(O)c2c(c1)C(=O)c1cccc(O)c1C2=O]

### Comparative Example 4

Basically the same as a preparation method of Example 1, the difference was: a raw material was methyl isoleucinate hydrochloride. A rhein amide derivative shown in formula V-4 was obtained as: methyl 2-(4,5-dihydroxy-9,10-dioxy-9,10-dihydroanthracene-2-carboxamide)-3-ethylvalerate, which was a yellow solid, with a yield (63 mg, 46.9%) and a melting point of 167.0° C. to 167.5° C.

¹H NMR (400 MHz, DMSO-d₆) δ 11.82 (s, 2H), 9.03 (d, J= 7.6 Hz, 1H), 8.06 (d, J= 1.7 Hz, 1H), 7.80 - 7.72 (m, 2H), 7.70 - 7.65 (m, 1H), 7.33 (dd, J = 8.3, 1.1 Hz, 1H), 4.30 (t, J = 7.5 Hz, 1H), 4.16 - 4.07 (m, 2H), 1.96 - 1.90 (m, 1H), 1.61 - 1.44 (m, 2H), 1.15 (d, J = 7.1 Hz, 3H), 0.86 (dd, J = 14.4, 7.1 Hz, 6H).

¹³C NMR (101 MHz, DMSO-d₆) δ 191.94, 181.55, 171.81, 165.36, 161.86, 161.50, 141.53, 138.06, 133.98, 133.75, 125.02, 123.36, 119.92, 118.49, 118.17, 116.55, 60.92, 58.12, 40.46, 36.08, 25.76, 19.12, 15.94, 14.61, 11.39.

HRMS (ESI+): molecular formula C₂₃H₂₃NO₇, [M+H]⁺426.1553, measured value: 426.1573.

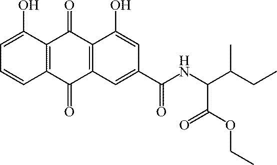 [SMILES: CCOC(=O)C(NC(=O)c1cc(O)c2c(c1)C(=O)c1cccc(O)c1C2=O)C(C)CC]

### Comparative Example 5

Basically the same as a preparation method of Example 1, the difference was: a raw material was ethyl phenylalaninate hydrochloride. A rhein amide derivative shown in formula V-5 was obtained as: ethyl (4,5-dihydroxy-9,10-dioxy-9,10-dihydroanthracene-2-carbonyl) phenylalaninate, which was a yellow solid, with a yield (79.5 mg, 54.8%) and a melting point of 171.0° C. to 172.7° C.

¹H NMR (400 MHz, DMSO-d₆) δ 12.12 - 11.63 (m, 2H), 9.33 (d, J = 7.7 Hz, 1H), 8.09 (d, J = 1.7 Hz, 1H), 7.82 (dd, J = 8.4, 7.5 Hz, 1H), 7.76 - 7.68 (m, 2H), 7.40 (dd, J = 8.3, 1.2 Hz, 1H), 7.35 - 7.26 (m, 4H), 7.24 - 7.18 (m, 1H), 4.82 - 4.56 (m, 1H), 4.12 (q, J = 7.1 Hz, 2H), 3.23 - 3.10 (m, 2H), 1.15 (t, J = 7.1 Hz, 3H).

¹³C NMR (101 MHz, DMSO-d₆) δ 171.76, 164.83, 161.84, 161.51, 141.25, 138.05, 137.93, 134.16, 129.55, 128.75, 127.04, 125.02, 123.00, 119.93, 118.35, 118.08, 116.58, 61.23, 55.01, 36.61, 14.47.

HRMS (ESI+): molecular formula C₂₆H₂₁NO₇, [M+H]⁺460.1396, measured value: 460.1399.

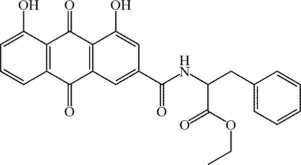 [SMILES: CCOC(=O)C(Cc1ccccc1)NC(=O)c1cc(O)c2c(c1)C(=O)c1cccc(O)c1C2=O]

### Comparative Example 6

Basically the same as a preparation method of Example 1, the difference was: a raw material was ethyl methioninate hydrochloride. A rhein amide derivative shown in formula V-6 was obtained as: ethyl(4,5-dihydroxy-9,10-dioxo-9,10-dihydroanthracene-2-carbonyl)methioninate, which was a yellow solid, with a yield (77 mg, 56.3%) and a melting point of 177.2° C. to 178.0° C.

¹H NMR (400 MHz, DMSO-d₆) δ 11.70 (s, 2H), 9.18 (d, J = 7.4 Hz, 1H), 8.09 (d, J = 1.7 Hz, 1H), 7.82 - 7.68 (m, 2H), 7.65 (d, J = 7.3 Hz, 1H), 7.32 (d, J = 8.3 Hz, 1H), 4.61 (td, J = 8.1, 6.0 Hz, 1H), 4.17 (tt, J = 7.2, 3.7 Hz, 2H), 2.70 - 2.54 (m, 2H), 2.18 - 2.10 (m, 2H), 2.09 (s, 3H), 1.22 (d, J = 7.0 Hz, 3H).

¹³C NMR (101 MHz, DMSO-d₆) δ 191.81, 181.26, 172.01, 164.95, 161.88, 161.60, 141.27, 138.00, 133.90, 133.56, 124.95, 123.20, 119.87, 118.22, 118.01, 116.30, 61.25, 52.53, 30.45, 30.38, 15.05, 14.56.

HRMS (ESI+): molecular formula C₂₂H₂₁N₇O₉, [M+H]⁺444.1117, measured value: 444.1128.

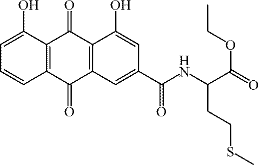 [SMILES: CCOC(=O)C(CCSC)NC(=O)c1cc(O)c2c(c1)C(=O)c1cccc(O)c1C2=O]

### Comparative Example 7

Basically the same as a preparation method of Example 1, the difference was: a raw material was ethyl valinate hydrochloride. A rhein amide derivative shown in formula V-7 was obtained as: ethyl(4,5-dihydroxy-9,10-dioxo-9,10-dihydroanthracene-2-carbonyl)valerate, which was a yellow solid, with a yield (90 mg, 69.3 %) and a melting point of 202.1° C. to 202.7° C.

¹H NMR (400 MHz, DMSO-d₆) δ 11.85 (s, 2H), 9.07 (d, J = 7.8 Hz, 1H), 8.11 (d, J = 1.7 Hz, 1H), 7.87 - 7.75 (m, 2H), 7.74 - 7.67 (m, 1H), 7.42 - 7.32 (m, 1H), 4.33 (t, J = 7.5 Hz, 1H), 4.17 (qq, J = 10.7, 7.0 Hz, 2H), 2.23 (dp, J = 13.1, 6.6 Hz, 1H), 1.24 - 1.20 (m, 3H), 1.00 (dd, J = 15.8, 6.8 Hz, 6H).

¹³C NMR (101 MHz, DMSO-d₆) δ 191.92, 181.42, 171.76, 165.37, 161.88, 161.52, 141.58, 138.03, 133.90, 133.69, 124.97, 123.37, 119.89, 118.52, 118.06, 116.46, 60.93, 59.38, 29.99, 19.55, 19.51, 14.62.

HRMS (ESI+): molecular formula C₂₂H₂₁NO₇, [M+H]⁺412.1396, measured value: 412.1420.

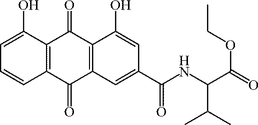 [SMILES: CCOC(=O)C(NC(=O)c1cc(O)c2c(c1)C(=O)c1cccc(O)c1C2=O)C(C)C]

### Comparative Example 8

Basically the same as a preparation method of Example 1, the difference was: a raw material was isobutylamine. A rhein amide derivative shown in formula V-8 was obtained as: 4,5-dihydroxy-N-isobutyl-9,10-dioxy-9,10-dihydroanthracene-2-carboxamide, which was a yellow solid, with a yield (34 mg, 31.7%) and a melting point of 278.2° C. to 279.1° C.

¹H NMR (400 MHz, DMSO-d₆) δ 11.91 (s, 2H), 8.91 (t, J = 5.8 Hz, 1H), 8.14 (d, J = 1.8 Hz, 1H), 7.84 (dd, J = 8.3, 7.5 Hz, 1H), 7.78 - 7.74 (m, 2H), 7.42 (dd, J = 8.4, 1.2 Hz, 1H), 3.12 (dd, J = 7.0, 5.8 Hz, 2H), 1.88 (dt, J = 13.5, 6.8 Hz, 1H), 0.91 (d, J = 6.6 Hz, 6H).

¹³C NMR (101 MHz, DMSO-d₆) δ 164.56, 161.87, 161.64, 142.46, 138.05, 134.09, 133.85, 125.02, 122.89, 119.93, 116.63, 90.50, 47.44, 28.46, 20.69.

HRMS (ESI+): molecular formula C₁₉H₁₇NO₅, [M+H]⁺340.1185, measured value: 340.1195. [0147]

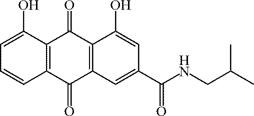 [SMILES: CC(C)CNC(=O)c1cc(O)c2c(c1)C(=O)c1cccc(O)c1C2=O]

### Comparative Example 9

Basically the same as a preparation method of Example 2, the difference was: a raw material was 4-methoxybenzylamine without triethylamine. A rhein amide derivative shown in formula V-9 was obtained as: 4,5-dihydroxy-N-(4-methoxybenzyl)-9,10-dioxy-9,10-dihydroanthracene-2-carboxamide, which was a yellow solid, with a yield (22.7 mg, 32%) and a melting point of 244.5° C. to 249.4° C.

¹H NMR (400 MHz, DMSO-d₆) δ 11.90 (s, 2H), 9.42 (t, J = 5.9 Hz, 1H), 8.17 (d, J = 1.7 Hz, 1H), 7.87 - 7.79 (m, 2H), 7.75 (dd, J = 7.5, 1.2 Hz, 1H), 7.41 (dd, J = 8.3, 1.2 Hz, 1H), 7.33 - 7.20 (m, 2H), 6.94 - 6.85 (m, 2H), 4.44 (d, J = 5.8 Hz, 2H), 3.74 (s, 3H).

¹³C NMR (101 MHz, DMSO-d₆) δ 192.00, 181.64, 164.34, 161.88, 161.65, 158.79, 142.10, 138.04, 134.13, 133.83, 131.53, 129.27, 125.01, 122.98, 119.93, 118.15, 118.08, 116.63, 114.23, 55.54, 42.88.

HRMS (ESI+): molecular formula C₂₃H₁₇NO₆, [M+H]⁺404.1134, measured value: 404.1138. [0153]

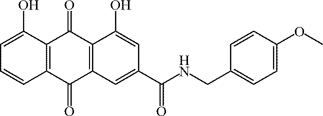 [SMILES: COc1ccc(CNC(=O)c2cc(O)c3c(c2)C(=O)c2cccc(O)c2C3=O)cc1]

### Comparative Example 10

Basically the same as a preparation method of Example 1, the difference was: a raw material was 4-piperazinoacetophenone. A rhein amide derivative shown in formula V-10 was obtained as: 3-(4-(4-acetylphenyl)piperazin-1-carbonyl)-1,8-dihydroxyanthracene-9,10-dione, which was a yellow solid, with a yield (33.8 mg, 40.8%) and a melting point of 270.1° C. to 272.2° C.

¹H NMR (400 MHz, Chloroform-d) δ 12.11 (s, 1H), 11.97 (s, 1H), 7.91 - 7.88 (m, 2H), 7.87 - 7.83 (m, 2H), 7.73 (dd, J= 8.4, 7.5 Hz, 1H), 7.36 (t, J= 1.1 Hz, 2H), 6.90 - 6.87 (m, 2H), 3.97 (s, 2H), 3.61 (s, 2H), 3.48 (s, 2H), 3.35 (s, 2H), 2.53 (s, 3H).

¹³C NMR (101 MHz, Chloroform-d) δ 196.54, 192.58, 181.01, 167.49, 162.79, 162.68, 153.58, 143.94, 137.70, 134.16, 133.35, 130.42, 128.76, 125.09, 122.62, 120.41, 117.93, 116.48, 115.71, 114.23, 48.06, 47.53, 47.02, 41.87, 29.71, 26.20.

HRMS (ESI+): molecular formula C₂₇H₂₂N₂O₆, [M+H]⁺471.1556, measured value: 471.1578.

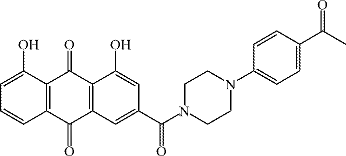 [SMILES: CC(=O)c1ccc(N2CCN(C(=O)c3cc(O)c4c(c3)C(=O)c3cccc(O)c3C4=O)CC2)cc1]

### Test Example

The effects of the above-prepared rhein amide derivatives on cell proliferation were detected by a CCK-8 kit.

The CCK-8 kit can evaluate a proliferation activity of cells by detecting an absorbance at a 450 nm wavelength. SNU398 and LO2 cells were digested to obtain a cell suspension, the cell suspension was diluted with a medium to a density of about 5×10³ cells/well, 100 µl of cell liquid was added to each well, and a blank group and a negative control group were set up. About 1.0000 mg of the derivative was added in a 0.5 mL EP tube, mixed with dimethyl sulfoxide (DMSO) to prepare a 5×10⁻² M stock solution, and stored at -20° C.

The stock solutions of 21 compounds prepared in the examples and comparative examples were diluted to final concentrations of 100 µmol/L, 20 µmol/L, 4 µmol/L, 0.8 µmol/L, and 0.16 µmol/L separately using a concentration gradient stepwise dilution method. An original medium in a 96-well plate was discarded by aspiration, 100 µl of the dilutions of the 17 compounds were added to the 96-well plate, and three parallel wells were set up. The dilutions were culture in a cell culture incubator for 48 h. A 10% CCK-8 test solution was prepared, after discarding the original drug solution by aspiration, the test solution was added at 100 µL per well into a 96-well plate; after culturing for 2 h, an OD value at a wavelength of 450 nm was measured, and an IC₅₀ value was calculated by data analysis to measure an inhibitory ability of the compounds on tumor cell proliferation. A stronger inhibitory ability led to a lower IC₅₀ value, and results were listed in Table 1; statistical analysis and drawing of experimental data was conducted using Graphpad-prism 6.0 software, and a mean ± standard deviation was expressed as (Mean ± S.E.M).

Table 1 Structures and inhibitory effects on proliferation of SNU-398 and LO2 cells of rhein amide derivatives prepared in Examples 1-7 and Comparative Examples 1-10

| No. | Name | Structure | Molecular weight | IC₅₀ (µM) | |
|---|---|---|---|---|---|
| | | | | SNU398 | LO2 |
| Comparative Example 1 | methyl (4,5-dihydroxy-9,10-dioxo-9, 10-dihydroanthracene-2-c arbonyl)leucine | 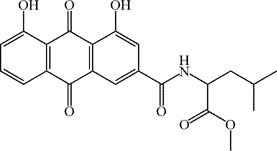 [SMILES: COC(=O)C(CC(C)C)NC(=O)c1cc(O)c2c(c1)C(=O)c1cccc(O)c1C2=O] | 411.41 | 25.90±6.94 | 0.81±0.16 |
| Comparative Example 2 | methyl 2-(4,5-dihydroxy-9,10-diox y-9,10-dihydroanthracene-2 -carboxamide)-3-methylval erate | 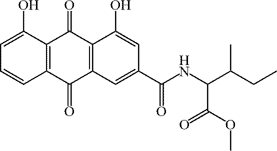 [SMILES: CCC(C)C(NC(=O)c1cc(O)c2c(c1)C(=O)c1cccc(O)c1C2=O)C(=O)OC] | 411.41 | 469.50±84.20 | 80.36±2.36 |
| Comparative Example 3 | ethyl(4,5-dihydroxy-9,10-di oxo-9,10-dihydroanthracene -2-carbonyl)leucinate | 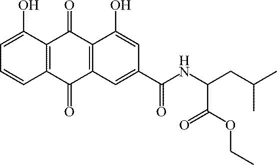 [SMILES: CCOC(=O)C(CC(C)C)NC(=O)c1cc(O)c2c(c1)C(=O)c1cccc(O)c1C2=O] | 425.43 | 188.50±94.40 | 57.13±3.25 |
| Comparative Example 4 | methyl 2-(4,5-dihydroxy-9,10-diox y-9,10-dihydroanthracene-2 -carboxamide)-3-ethylvaler ate | 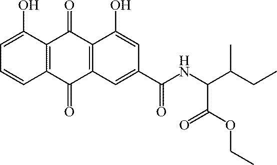 [SMILES: CCOC(=O)C(NC(=O)c1cc(O)c2c(c1)C(=O)c1cccc(O)c1C2=O)C(C)CC] | 425.43 | 47.12±11.49 | 18.21±1.56 |
| Comparative Example 5 | ethyl (4,5-dihydroxy-9,10-dioxy-9, 10-dihydroanthracene-2-c arbonyl) phenylalaninate | 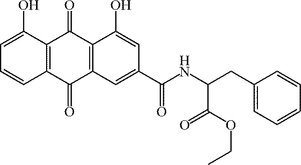 [SMILES: CCOC(=O)C(Cc1ccccc1)NC(=O)c1cc(O)c2c(c1)C(=O)c1cccc(O)c1C2=O] | 459.45 | 165.80±7.62 | 228.50±7.25 |
| Example 3 | diethyl(4,5-dihydroxy-9,10-dioxo-9,10-dihydroanthrace ne-2-carbonyl) glutamate | 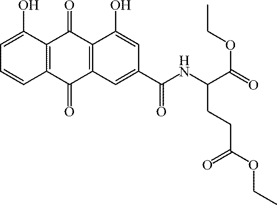 [SMILES: CCOC(=O)CCC(NC(=O)c1cc(O)c2c(c1)C(=O)c1cccc(O)c1C2=O)C(=O)OCC] | 469.44 | 66.77±2.21 | 113.90±4.11 |
| Comparative Example 6 | ethyl(4,5-dihydroxy-9,10-di oxo-9,10-dihydroanthracene -2-carbonyl)methioninate | 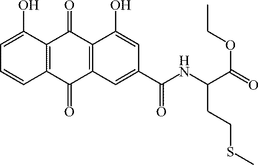 [SMILES: CCOC(=O)C(CCSC)NC(=O)c1cc(O)c2c(c1)C(=O)c1cccc(O)c1C2=O] | 443.47 | 19.78±1.23 | 7.77±0.45 |
| Comparative Example 7 | ethyl(4,5-dihydroxy-9,10-di oxo-9,10-dihydroanthracene -2-carbonyl)valerate | 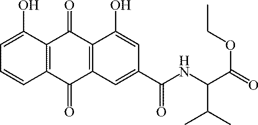 [SMILES: CCOC(=O)C(NC(=O)c1cc(O)c2c(c1)C(=O)c1cccc(O)c1C2=O)C(C)C] | 411.41 | 160.90±4.53 | 13.73±0.89 |
| Example 1 | methyl(4,5-dihydroxy-9,10-dioxy-9,10-dihydroanthrace ne-2-carbonyl) alaninate | 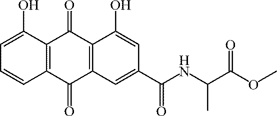 [SMILES: COC(=O)C(C)NC(=O)c1cc(O)c2c(c1)C(=O)c1cccc(O)c1C2=O] | 369.08 | 0.46±0.08 | 5.41+0.55 |
| Example 4 | diethyl(4,5-dihydroxy-9,10-dioxo-9,10-dihydroanthrace ne-2-carbonyl)aspartate | 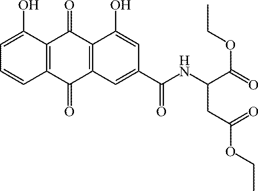 [SMILES: CCOC(=O)CC(NC(=O)c1cc(O)c2c(c1)C(=O)c1cccc(O)c1C2=O)C(=O)OCC] | 455.12 | 27.90±3.45 | 117.20+5.65 |
| Example 2 | ethyl 2-(4,5-dihydroxy-9,10-diox y-9,10-dihydroanthracene-2 -carboxamide)butanoate | 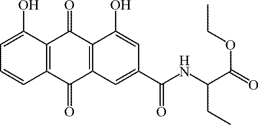 [SMILES: CCOC(=O)C(CC)NC(=O)c1cc(O)c2c(c1)C(=O)c1cccc(O)c1C2=O] | 397.12 | 8.81±0.78 | 103.80±9.36 |
| Example 5 | 4,5-dihydroxy-9,10-dioxo-n -propyl-9, 10-dihydroanthra cene-2-carboxamide | 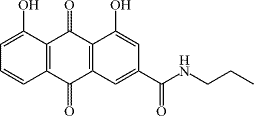 [SMILES: CCCNC(=O)c1cc(O)c2c(c1)C(=O)c1cccc(O)c1C2=O] | 325.10 | 116.30±5.25 | 228.20±10.80 |
| Example 6 | N-butyl-4,5-dihydroxy-9,10 -dioxy-9,10-dihydroanthrac ene-2-carboxamide | 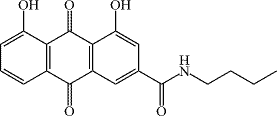 [SMILES: CCCCNC(=O)c1cc(O)c2c(c1)C(=O)c1cccc(O)c1C2=O] | 339.11 | 5.85±0.49 | 113.10±8.56 |
| Comparative Example 8 | 4,5-dihydroxy-N-isobutyl-9, 1 0-dioxy-9, 10-dihydroanthr acene-2-carboxamide | 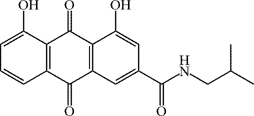 [SMILES: CC(C)CNC(=O)c1cc(O)c2c(c1)C(=O)c1cccc(O)c1C2=O] | 339.11 | --- | 118.40±12.30 |
| Comparative Example 9 | 4,5-dihydroxy-N-(4-methox ybenzyl)-9,10-dioxy-9,10-di hydroanthracene-2-carboxa mide | 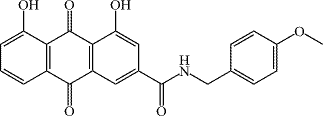 [SMILES: COc1ccc(CNC(=O)c2cc(O)c3c(c2)C(=O)c2cccc(O)c2C3=O)cc1] | 403 | 110.70±12.45 | 106.50±9.72 |
| Comparative Example 10 | 3-(4-(4-acetylphenyl)pipera zin-1-carbonyl)-1,8-dihydro xyanthracene-9,10-dione | 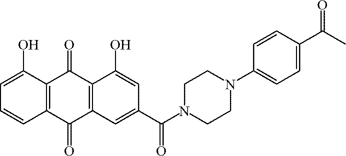 [SMILES: CC(=O)c1ccc(N2CCN(C(=O)c3cc(O)c4c(c3)C(=O)c3cccc(O)c3C4=O)CC2)cc1] | 470.48 | 221.50±18.68 | 104.30±9.96 |
| Example 7 | ethyl (4-(4,5-dihydroxy-9,10-diox y-9,10-dihydroanthracene-2 -carbonyl)piperazin-1-yl)be nzoate | 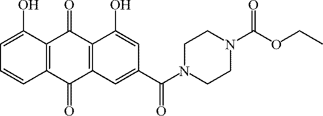 [SMILES: CCOC(=O)N1CCN(C(=O)c2cc(O)c3c(c2)C(=O)c2cccc(O)c2C3=O)CC1] | 424.12 | 19.03±1.96 | 26.31±15.82 |

The structures and inhibitory effects on proliferation of SNU-398 and LO2 cells of rhein amide derivatives prepared in Examples 1-7 and Comparative Examples 1-10 listed in Table 1 show that the IC₅₀ of the rhein amide derivatives prepared in Examples 1-7 on HCC cells SNU398 is less than the IC₅₀ on hepatocytes LO2. The IC₅₀ of the rhein amide derivatives prepared in Example 1, Example 2, Example 4 and Example 6 on HCC cells SNU398 is much smaller than the IC₅₀ on hepatocytes LO2, indicating that the rhein amide derivatives prepared in the examples have a stronger inhibitory effect on the proliferation of HCC cells SNU398 with high RECQL4 expression compared with the rhein amide derivatives prepared in Comparative Examples 1-11. In addition, compared with the IC₅₀ of hepatocytes LO2, it shows that these derivatives each have a selective inhibitory effect on the proliferation of HCC cells SNU398 with high RECQL4 expression.

Although the above embodiments have described the present disclosure in a thorough manner, it is only some but not all embodiments of the present disclosure, and other embodiments may be obtained without inventive step according to the present embodiments, all of which fall within the scope of protection the present disclosure.

What is claimed is:

1. A rhein amide derivative, having a structure shown in any one of formulas I to IV: 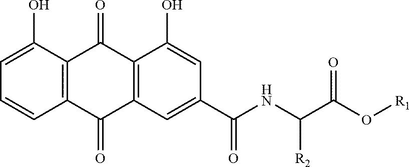 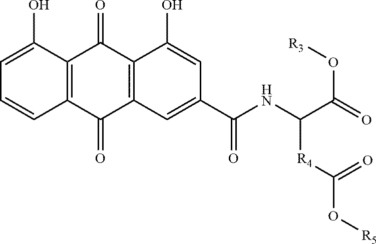 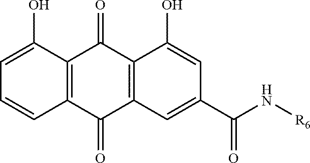 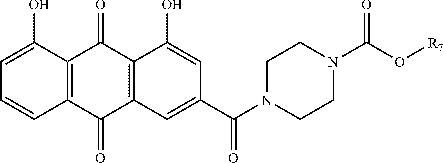
; wherein
R₁, R₂, R₃, R₄, R₅, and R₇ are independently selected from the group consisting of methyl and ethyl; and R₆ is linear C₁₋₄ alkyl.

2. The rhein amide derivative according to claim 1, having a structure shown in formula I-1, formula I-2, formula II-1, formula II-2, formula III-1, formula III-2, or formula IV-1: 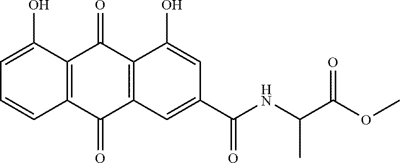 [SMILES: COC(=O)C(C)NC(=O)c1cc(O)c2c(c1)C(=O)c1cccc(O)c1C2=O] 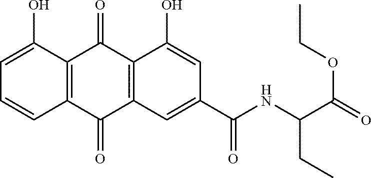 [SMILES: CCOC(=O)C(CC)NC(=O)c1cc(O)c2c(c1)C(=O)c1cccc(O)c1C2=O] 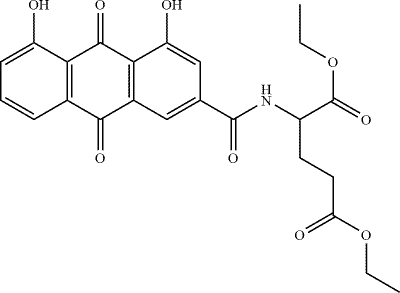 [SMILES: CCOC(=O)CCC(NC(=O)c1cc(O)c2c(c1)C(=O)c1cccc(O)c1C2=O)C(=O)OCC] 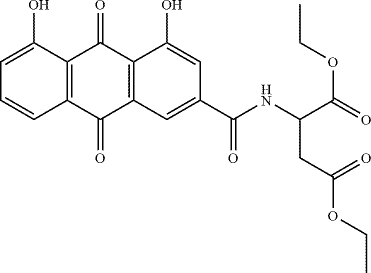 [SMILES: CCOC(=O)CC(NC(=O)c1cc(O)c2c(c1)C(=O)c1cccc(O)c1C2=O)C(=O)OCC] 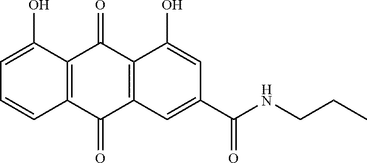 [SMILES: CCCNC(=O)c1cc(O)c2c(c1)C(=O)c1cccc(O)c1C2=O] 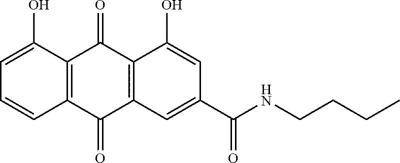 [SMILES: CCCCNC(=O)c1cc(O)c2c(c1)C(=O)c1cccc(O)c1C2=O]
and 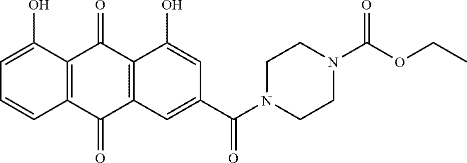 [SMILES: CCOC(=O)N1CCN(C(=O)c2cc(O)c3c(c2)C(=O)c2cccc(O)c2C3=O)CC1]
formula IV-1.

3. A method of preparing the rhein amide derivative according to claim 1, comprising:
mixing rhein, any one of a reactant 1 to a reactant 3, a coupling agent, an acylation catalyst, an organic base, and an organic solvent to conduct an amidation reaction, to obtain the rhein amide derivative having a structure shown in any one of formulas I to III; and
mixing the rhein, a reactant 4, the coupling agent, the acylation catalyst, and the organic solvent to conduct the amidation reaction, to obtain a rhein amide derivative having a structure shown in formula V or formula VI; wherein
the reactant 1 is a hydrochloride of 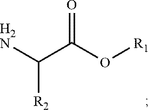
the reactant 2 is a hydrochloride of 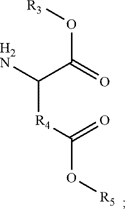
the reactant 3 is 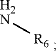
and the reactant 4 is 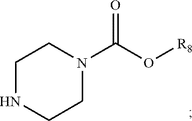
and
R₁, R₂, R₃, R₄, R₅, and R₇ are independently selected from the group consisting of methyl and ethyl; and R₆ is linear C₁₋₄ alkyl.

4. The method according to claim 2, comprising:
mixing rhein, any one of a reactant 1 to a reactant 3, a coupling agent, an acylation catalyst, an organic base, and an organic solvent to conduct an amidation reaction, to obtain the rhein amide derivative having a structure shown in any one of formulas I to III; and
mixing the rhein, a reactant 4, the coupling agent, the acylation catalyst, and the organic solvent to conduct the amidation reaction, to obtain a rhein amide derivative having a structure shown in formula V or formula VI; wherein
the reactant 1 is a hydrochloride of 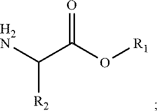
the reactant 2 is a hydrochloride of 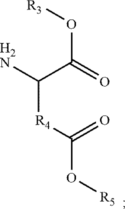
the reactant 3 is 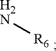
and the reactant 4 is 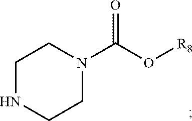
and
R₁, R₂, R₃, R₄, R₅, and R₇ are independently selected from the group consisting of methyl and ethyl; and R₆ is linear C₁₋₄ alkyl.

5. The method according to claim 3, wherein the rhein and any one of the reactant 1 to the reactant 4 have a molar ratio of 1:(1.0-1.5).

6. The method according to claim 4, wherein the rhein and any one of the reactant 1 to the reactant 4 have a molar ratio of 1:(1.0-1.5).

7. The method according to claim 3, wherein the rhein and the coupling agent have a molar ratio of 1:(1.8-2.5).

8. The method according to claim 4, wherein the rhein and the coupling agent have a molar ratio of 1:(1.8-2.5).

9. The method according to claim 3, wherein the rhein and the acylation catalyst have a molar ratio of 1:(1.8-2.3).

10. The method according to claim 4, wherein the rhein and the acylation catalyst have a molar ratio of 1:(1.8-2.3).

11. The method according to claim 3, wherein the rhein and the organic base have a molar ratio of 1:(2.5-4.5).

12. The method according to claim 4, wherein the rhein and the organic base have a molar ratio of 1:(2.5-4.5).

13. The method according to claim 3, wherein the amidation reaction is conducted at room temperature for 1.8 h to 7 h.

14. The method according to claim 4, wherein the amidation reaction is conducted at room temperature for 1.8 h to 7 h.

15. The method according to claim 5, wherein the amidation reaction is conducted at room temperature for 1.8 h to 7 h.

16. The method according to claim 6, wherein the amidation reaction is conducted at room temperature for 1.8 h to 7 h.

17. The method according to claim 7, wherein the amidation reaction is conducted at room temperature for 1.8 h to 7 h.

18. The method according to claim 8, wherein the amidation reaction is conducted at room temperature for 1.8 h to 7 h.

19. The method according to claim 9, wherein the amidation reaction is conducted at room temperature for 1.8 h to 7 h.

20. An inhibitor for inhibiting overexpression of RECQL4, comprising the rhein amide derivative according to claim 1.
